(19) United States
(12) Patent Application Publication
(10) Pub. No.: US 2026/0335529 A1
(43) Pub. Date: Oct. 29, 2026

(54) NASAL ASPIRATOR AND ITS FILTER AIR PASSAGE ASSEMBLY AND SUCTIONING TIP ASSEMBLY

(71) Applicant: Shenzhen Dongjiang Technology Co., Ltd., Shenzhen (CN)

(72) Inventor: JUNJIE WEI, Gongyi (CN)

(21) Appl. No.: 19/707,111

(22) Filed: Jun. 12, 2026

(30) Foreign Application Priority Data
Jan. 15, 2026 (CN) 202620052749.9
Jan. 15, 2026 (CN) 202620053760.7

(51) Int. Cl. A61M 1/00; A61B 1/06; A61B 1/233; A61B 17/24

(52) U.S. Cl. CPC A61M 1/87; A61B 1/06; A61B 1/233; A61B 17/24; A61M 1/79; A61B 2017/246

(57) ABSTRACT

The present invention proposes a nasal aspirator, its filter air passage assembly having a fixing casing, an air passage piece, and a conversion cap, and its suctioning tip assembly. The air passage piece is fixedly connected inside the fixing casing, and the conversion cap forms a sealed and fixed connection together with the fixing casing and covers the air passage piece. The air passage piece has an air-suctioning hole communicating with a first airflow chamber, and an air-expelling hole communicating with a second airflow chamber, both communicating with external environments through the fixing casing. The conversion cap has an air-suctioning end communicating with the first airflow chamber and staggering the air-suctioning hole, and an air-expelling end communicating with the second airflow chamber and staggering the air-expelling hole. It effectively prevents foreign matter in the nasal cavity from being suctioned into the air pump and avoiding the air passage from cogging.

### CROSS-REFERENCE TO RELATED PATENT APPLICATION

The present application claims priority to Chinese Patent Application No. CN 202620053760.7, filed on Jan. 15, 2026, and Chinese Patent Application No. CN 202620052749.9, filed on Jan. 15, 2026, the entire disclosure of which is incorporated herein by reference in its entirety.

### FIELD OF THE INVENTION

The present invention relates to the technical field of nasal aspirator accessories, and in particular to a filter air passage assembly for nasal aspirators, an easily-replaceable integrated suctioning tip assembly, and a nasal aspirator.

### BACKGROUND OF THE INVENTION

When children have a cold and a stuffy nose, they cannot suction out their own mucus; therefore, the stuffy nose may lead to difficulty breathing and affect children's sleep. A nasal aspirator aids children in removing secretions from nasal cavities and breathing freely. It is difficult for parents to manually operate traditional nasal aspirators and suction the secretions out of children's nasal cavities. In case of applying a too big suction force, they easily injure children's nasal cavities.

Existing automatic nasal aspirators typically include a collection assembly, and a main body provided with an air pump that generates a negative pressure to suction mucus out of children's nasal cavities. In general, the part where the collection assembly is joined to the main body is directly connected with the air pump, and a separate space is usually designed respectively for the collection assembly and the main body to prevent mucus from being suctioned back. However, during suctioning, it is easy to suction out foreign matter in the nasal cavity (such as nasal hair, booger, etc.). Since this foreign matter is relatively light, if its size amounts to the diameter of the air pump's air passage, it will be directly suction into the air pump along the air passage and get stuck in the air pump, causing damage to the air pump. If the size of this foreign matter is smaller than the diameter of the air pump's air passage, it will slide along the air passage, and directly slide out without affecting the air pump, in case of sliding smoothly. However, the foreign matter that has undergone sliding for long time will dehydrate, then easily sticks to an inner wall of a negative pressure channel, causing airflow obstruction in the negative pressure channel; in severe cases, it will reduce the efficiency of the air pump and go against the normal use of the nasal aspirator.

For this reason, it is necessary to propose a filter air passage assembly for nasal aspirators to prevent foreign matter in the nasal cavity from being suctioned out and getting into the air pump, while also avoiding the air passage from clogging.

### SUMMARY OF THE INVENTION

In order to solve the above problems, the present invention provides a filter air passage assembly for nasal aspirators, capable of preventing foreign matter in the nasal cavity from being suctioned out and getting into the air pump, while also avoiding the air passage from clogging.

In order to achieve the above object, the present invention adopts the following technical solutions:
The present invention provides a filter air passage assembly for nasal aspirators, configured to be fixedly mounted on one end of a main body of the nasal aspirator, and joined to a suctioning tip assembly;
comprising a fixing casing, an air passage piece, and a conversion cap; in which the air passage piece is fixedly connected inside the fixing casing, and the conversion cap forms a sealed and fixed connection together with the fixing casing and covers the air passage piece;
an air-suctioning hole, a first airflow chamber, a second airflow chamber, and an air-expelling hole are disposed on the air passage piece, and the air-suctioning hole and the air-expelling hole both communicate with external environments through the fixing casing, and the air-suctioning hole communicates with the first airflow chamber, and the air-expelling hole communicates with the second airflow chamber;
an air-suctioning end and an air-expelling end are disposed on the conversion cap, and the air-suctioning end communicates with the first airflow chamber and staggers the air-suctioning hole, and the air-expelling end communicates with the second airflow chamber and staggers the air-expelling hole.

In some examples, the air-suctioning hole is arranged on one side of the top wall of the first airflow chamber, while the air-suctioning end is arranged on one side of the bottom wall of the first airflow chamber; or, the air-expelling hole is arranged on one side of the top wall of the second airflow chamber, while the air-expelling end is arranged on one side of the bottom wall of the second airflow chamber.

In some examples, the air-suctioning hole is arranged on one side of the top wall of the first airflow chamber, while the air-suctioning end is arranged on one side of the bottom wall of the first airflow chamber; and, the air-expelling hole is arranged on one side of the top wall of the second airflow chamber, while the air-expelling end is arranged on one side of the bottom wall of the second airflow chamber.

In some examples, the volume of the first airflow chamber is a, while the volume of the second airflow chamber is b, conforming to a>b.

In some examples, an installation groove is disposed at one end of the fixing casing, and the air passage piece is accommodated in the installation groove; a transition groove is disposed at the other end of the fixing casing, and configured to mount one end of the suctioning tip assembly, and the air-suctioning hole and the air-expelling hole both communicate with the transition groove.

In some examples, a plug-in lip is disposed at one end of the air passage piece, and the plug-in lip penetrates the fixing casing, and the air-suctioning hole communicates with the transition groove through the plug-in lip.

In some examples, an embedded ring groove is disposed at one end of the fixing casing, and an embedding protrusion ring is disposed on one side of the conversion cap, and the embedding protrusion ring fits into the embedded ring groove and makes a tight join on the wall of the embedded ring groove.

In some examples, the filter air passage assembly for nasal aspirators further comprises an illuminating bead and an accommodating groove disposed on the air passage piece, and the illuminating bead is embedded in the accommodating groove, and a second evading hole is disposed on the fixing housing, and the second evading hole communicates with a transition groove, and the illuminating bead is aligned with the second evading hole.

In some examples, the air passage piece is made from soft materials.

In some examples, the first airflow chamber extends in its length direction, and the air-suctioning hole and the air-suctioning end are respectively arranged at the two ends of the first airflow chamber in its length direction; or the second airflow chamber extends in its length direction, and the air-expelling hole and the air-expelling end are respectively arranged at the two ends of the second airflow chamber in its length direction.

In some examples, the first airflow chamber extends in its length direction, and the air-suctioning hole and the air-suctioning end are respectively arranged at the two ends of the first airflow chamber in its length direction; and the second airflow chamber extends in its length direction, and the air-expelling hole and the air-expelling end are respectively arranged at the two ends of the second airflow chamber in its length direction.

In some examples, the filter air passage assembly for nasal aspirators is configured to be connected with an easily-replaceable integrated suctioning tip assembly, which comprises a main body and a suctioning tip assembly;

one end of the suctioning tip assembly is detachably connected and communicates with one end of the main body, and the suctioning tip assembly includes a connecting communicating seat, a collecting piece, and a suction nozzle; one end of the connecting communicating seat fits and communicates with one end of the main body, and the other end of the connecting communicating seat is detachably connected and communicates with the collecting piece; one end of the suction nozzle forms a detachable sealed connection with the periphery of the connecting communicating seat, and the suction nozzle envelops the collecting piece to form a negative pressure space, and the suction nozzle communicates with the negative pressure space, so as to make it possible to store foreign matter suctioned by the suctioning tip assembly in the collecting piece, and ensure that when the foreign matter in the collecting piece overflows, it falls into the connecting communicating seat.

In the present invention, the fixing casing serves as a structure connected with the collection assembly, and the air passage piece serves as a piece for filtering airflow during suctioning. The air-suctioning end and the air-expelling end arranged on the conversion cap are respectively connected with the air-suctioning port and the air-expelling port of the air pump. The airflow passes out of alignment by enabling the air-suctioning end to communicate with the first airflow chamber while staggering the air-suctioning hole, and enabling the air-expelling end to communicate with the second airflow chamber while staggering the air-expelling hole, so as to make it possible for any foreign matter suctioned out in spite of getting into the air passage piece to stay in the first airflow chamber or the second airflow chamber under the obstruction of these chambers, and avoid a blockage in the original air passage, and larger particles of foreign matter from getting into the air pump. In summary, the filter air passage assembly for nasal aspirators can effectively prevent foreign matter in the nasal cavity from being suctioned into the air pump and avoiding the air passage from cogging.

The present invention further provides a nasal aspirator, comprising a main body, a suctioning tip assembly and a filter air passage assembly having a fixing casing, an air passage piece, and a conversion cap;
the air passage piece is fixedly connected inside the fixing casing, and the conversion cap forms a sealed and fixed connection together with the fixing casing and covers the air passage piece;
an air-suctioning hole, a first airflow chamber, a second airflow chamber, and an air-expelling hole are disposed on the air passage piece, and the air-suctioning hole and the air-expelling hole both communicate with external environments through the fixing casing, and the air-suctioning hole communicates with the first airflow chamber, and the air-expelling hole communicates with the second airflow chamber;
an air-suctioning end and an air-expelling end are disposed on the conversion cap, and the air-suctioning end communicates with the first airflow chamber and staggers the air-suctioning hole, and the air-expelling end communicates with the second airflow chamber and staggers the air-expelling hole;
the fixing casing is fixedly mounted on one end of the main body, and the fixing casing is configured as a structure connected with the suctioning tip assembly;
the suctioning tip assembly communicates with the air passage piece, and an air pump is disposed inside the main body, and the air-suctioning end and the air-expelling end respectively communicate with an air-suctioning port and an air-expelling port of the air pump.

In some examples, one end of the suctioning tip assembly is detachably connected and communicates with one end of the main body, and the suctioning tip assembly includes a connecting communicating seat, a collecting piece, and a suction nozzle; one end of the connecting communicating seat fits and communicates with one end of the main body, and the other end of the connecting communicating seat is detachably connected and communicates with the collecting piece; one end of the suction nozzle forms a detachable sealed connection with the periphery of the connecting communicating seat, and the suction nozzle envelops the collecting piece to form a negative pressure space, and the suction nozzle communicates with the negative pressure space, so as to make it possible to store foreign matter suctioned by the suctioning tip assembly in the collecting piece, and ensure that when the foreign matter in the collecting piece overflows, it falls into the connecting communicating seat.

In some examples, a mounting portion is disposed at one end of the connecting communicating seat, and an engagement ventilation portion is disposed at one end of the main body, and the mounting portion is accommodated in the engagement ventilation portion and forms an engagement connection with the engagement ventilation portion, and the mounting portion communicates with the engagement ventilation portion.

In some examples, the mounting portion is provided with an inserted conduit column, and the engagement ventilation portion is provided with an air-suctioning groove, and one end of the inserted conduit column is accommodated in the air-suctioning groove, and the outer wall of the inserted conduit column closely fits onto the wall of the air-suctioning groove.

In some examples, a plug-in sleeve is disposed on the collecting piece, and the plug-in sleeve forms a detachable connection and communicates with the other end of the inserted conduit column, and a through hole is disposed on a side wall of the plug-in sleeve, and the through hole communicates with the negative pressure space.

In some examples, the mounting portion is provided with a caught side ring, and the engagement ventilation portion is provided with an embedded catch groove, and the caught side ring fits into the embedded catch groove and forms an engagement connection with the wall of the embedded catch groove.

In some examples, a caught plate is disposed on the periphery of the caught side ring, and a catch opening is disposed on the wall of the embedded catch groove, and the caught plate fits into the catch opening.

In some examples, a suction tube is disposed inside the suction nozzle, and a collecting groove is disposed at one end of the collecting piece, and one end of the suction tube extends into the collecting groove, and a plurality of guiding grooves are disposed at one end of the collecting piece, and circumferentially uniformly distributed, and the plurality of guiding grooves communicate with the collecting groove, and also communicate with the negative pressure space, and the suction tube communicates with the negative pressure space through the plurality of guiding grooves.

In some examples, a plurality of collecting tips are disposed at one end of the suction tube, and extend into the collecting groove.

In some examples, an anti-slip portion for improving grip strength is disposed on the periphery of the connecting communicating seat.

The filter air passage assembly for nasal aspirators of the present invention can effectively prevent foreign matter in the nasal cavity from being suctioned into the air pump and avoiding the air passage from cogging. Meantime, by way of providing the suctioning tip assembly composed of the connecting communicating seat, the collecting piece, and the suction nozzle and the detachable connection formed by the connecting communicating seat and the main body, when it is necessary to clean the suctioning tip assembly, users can directly detach the suctioning tip assembly from the main body. Since the suctioning tip assembly is an integrated unit, the collected mucus falls into the suctioning tip assembly, preventing contamination on the main body. When cleaning the interior of the suctioning tip assembly, users can directly disassemble the suctioning tip assembly into the connecting communicating seat, the collecting piece, and the suction nozzle for respective cleaning, with advantages such as convenience and usefulness. In summary, the easily-replaceable integrated suctioning tip assembly can be disassembled together with a part connected with a suctioning tip, making subsequent cleaning more convenient.

### BRIEF DESCRIPTION OF THE DRAWINGS

FIG. 1 is an exploded view of the filter air passage assembly for nasal aspirators according to the present invention when installed in the main body of the nasal aspirator.

FIG. 2 is an exploded view of the filter air passage assembly for nasal aspirators according to the present invention.

FIG. 3 is a diagram depicting the internal structure of the filtrating air passage assembly for nasal aspirators according to the present invention.

FIG. 4 is an overall diagram of the filter air passage assembly for nasal aspirators according to the present invention.

FIG. 5 is a diagram of the conversion cap of the filter air passage assembly for nasal aspirators according to the present invention.

FIG. 6 is a diagram of the air passage piece of the filter air passage assembly for nasal aspirators according to the present invention.

FIG. 7 is a diagram of the air passage piece of the filter air passage assembly for nasal aspirators according to the present invention, from another viewpoint.

FIG. 8 is an exploded view of installing the easily-replaceable integrated suctioning tip assembly on the main body in the present invention.

FIG. 9 is a partial enlarged view of A in FIG. 8.

FIG. 10 is an exploded view of the easily-replaceable integrated suctioning tip assembly according to the present invention.

FIG. 11 is a diagram of the easily-replaceable integrated suctioning tip assembly according to the present invention.

FIG. 12 is a partial enlarged view of B in FIG. 11.

FIG. 13 is a sectional view of the easily-replaceable integrated suctioning tip assembly according to the present invention.

### DESCRIPTION OF SYMBOLS

1—fixing casing; 11—installation groove; 12—transition groove; 13—first evading hole; 14—embedded ring groove; 15—second evading hole; 2—air passage piece; 21—air-suctioning hole; 22—first airflow chamber; 23—second airflow chamber; 24—air-expelling hole; 25—plug-in lip; 26—accommodating groove; 3—conversion cap; 31—air-suctioning end; 32—air-expelling end; 33—embedding protrusion ring; 34—first sealing ring portion; 35—second sealing ring portion; 4—illuminating bead; 5—air pump; 51—air-suctioning port; 52—air-expelling port; 6—suctioning tip assembly; 61—connecting communicating seat; 611—mounting portion; 6111—inserted conduit column; 6112—caught side ring; 61121—caught plate; 601—positioning groove; 612—anti-slip portion; 62—collecting piece; 621—plug-in sleeve; 6211—through hole; 622—collecting groove; 623—guiding groove; 63—suction nozzle; 631—negative pressure space; 632—suction tube; 6321—collecting tip; 633—suction nozzle end; 7—main body; 71—engagement ventilation portion; 711—air-suctioning groove; 712—embedded catch groove; 7121—catch opening; 701—positioning protrusion structure.

### Detailed Description of Some Embodiments

In order to more clearly and completely illustrate the technical solution of the present invention, we shall further describe the present invention in combination with the figures as follows.

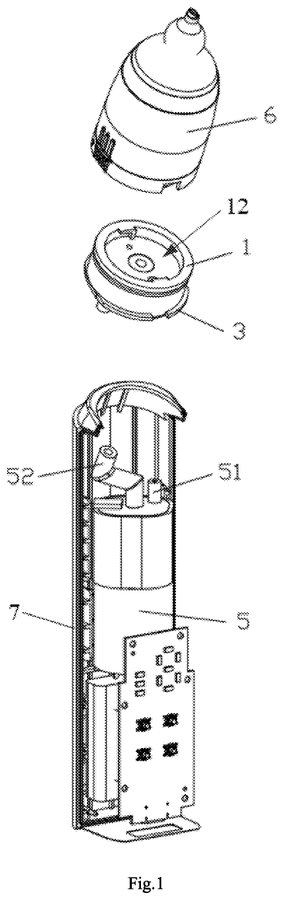

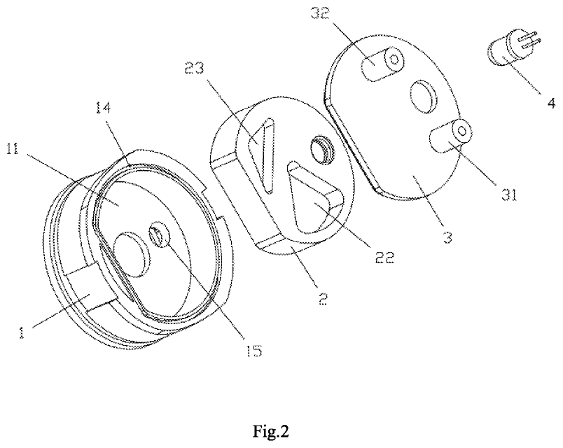

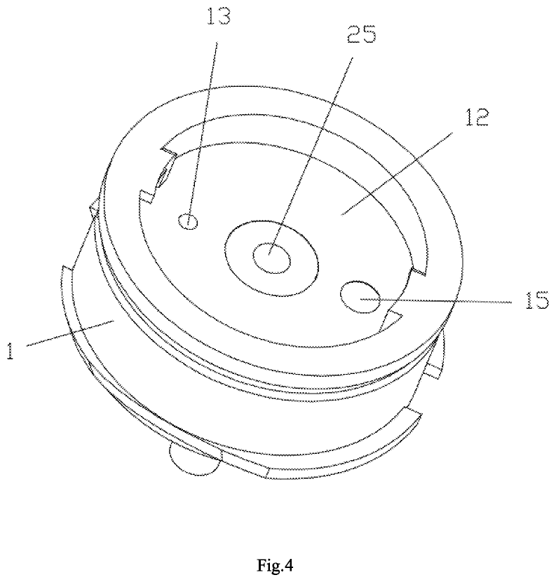

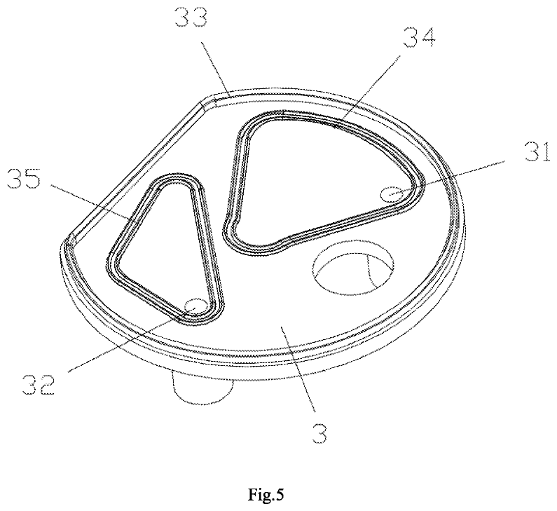

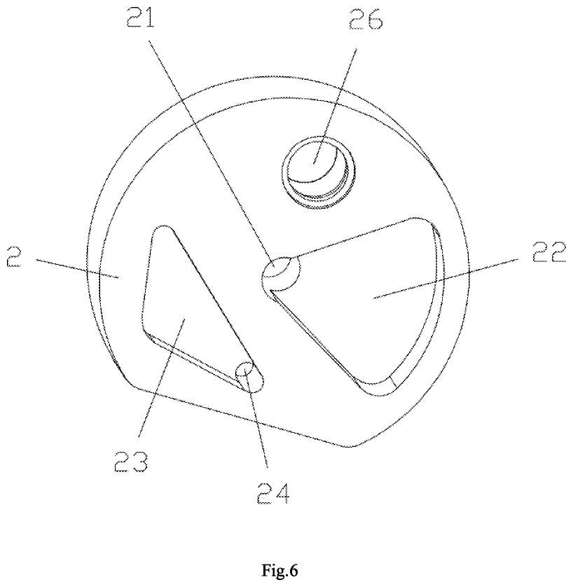

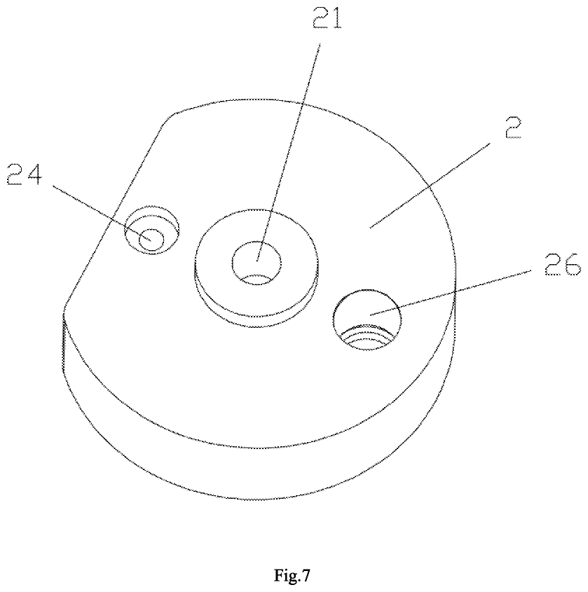

As shown FIGS. 1-7, a filter air passage assembly for nasal aspirators according to the present invention, comprises a fixing casing 1, an air passage piece 2, and a conversion cap 3. The air passage piece 2 is fixedly connected inside the fixing casing 1 and is made from soft materials, such as silicone materials. The conversion cap 3 forms a sealed and fixed connection together with the fixing casing 1 and covers the air passage piece 2. An air-suctioning hole 21, a first airflow chamber 22, a second airflow chamber 23, and an air-expelling hole 24 are disposed on the air passage piece 2. The air-suctioning hole 21 and the air-expelling hole 24 both communicate with external environments through the fixing casing 1. The air-suctioning hole 21 communicates with the first airflow chamber 22, and the air-expelling hole 24 communicates with the second airflow chamber 23. An air-suctioning end 31 and an air-expelling end 32 are disposed on the conversion cap 3. The air-suctioning end 31 communicates with the first airflow chamber 22 and staggers the air-suctioning hole 21. The air-expelling end 32 communicates with the second airflow chamber 23 and staggers the air-expelling hole 24.

In this embodiment, the fixing casing 1 serves as a structure connected with a suctioning tip assembly 6, and the fixing casing 1 is fixedly mounted on one end of the main body of the nasal aspirator. After the suctioning tip assembly 6 is mounted on the fixing casing 1, the suctioning tip assembly 6 communicates with the air passage piece 2, and a negative pressure is transmitted to the suctioning tip assembly 6 through the air passage piece 2. The air passage piece 2 serves as a piece for filtering airflow during suctioning of the suctioning tip assembly 6. The air-suctioning end 31 and the air-expelling end 32 arranged on the conversion cap 3 are respectively connected with the air-suctioning port 51 and the air-expelling port 52 of the air pump 5. After the air pump 5 starts, the air-suctioning port 51 begins to suction air, and the air-expelling port 52 also starts to discharge the suctioned air. The airflow passes out of alignment by enabling the air-suctioning end 31 to communicate with the first airflow chamber 22 while staggering the air-suctioning hole 21, and enabling the air-expelling end 32 to communicate with the second airflow chamber 23 while staggering the air-expelling hole 24, so as to make it possible for any foreign matter suctioned out in spite of getting into the air passage piece 2 to stay in the first airflow chamber 22 or the second airflow chamber 23 under the obstruction of these chambers, and avoid a blockage in the original air passage, and larger particles of foreign matter from getting into the air pump 5.

In summary, the filter air passage assembly for nasal aspirators can effectively prevent foreign matter in the nasal cavity from being suctioned into the air pump and avoiding the air passage from cogging.

In this embodiment, the air-suctioning hole 21 is arranged on one side of the top wall of the first airflow chamber 22, and the air-suctioning end 31 is arranged on one side of the bottom wall of the first airflow chamber 22; thus, by way of arranging the air-suctioning hole 21 and the air-suctioning end 31 at different positions of the first airflow chamber 22, respectively, it is possible for any foreign matter in spite of getting suctioned into the first airflow chamber 22 to stay in the first airflow chamber 22, preventing itself from getting suctioned into the air pump 5.

In this embodiment, the volume of the first airflow chamber 22 is a, while the volume of the second airflow chamber 23 is b, conforming to a>b. The first airflow chamber 22 serves as a chamber which the foreign matter first gets into on being suctioned; therefore, it is necessary to create a larger space to make more possible to make the foreign matter stay. In addition, even if the foreign matter stays in the first airflow chamber 22, it is still necessary to ensure smooth airflow. In comparison, the second airflow chamber 23 serves as the last barrier for making the foreign matter stay. The combination of the first airflow chamber 22 and the second airflow chamber 23 ensures the smoothness of the air passage.

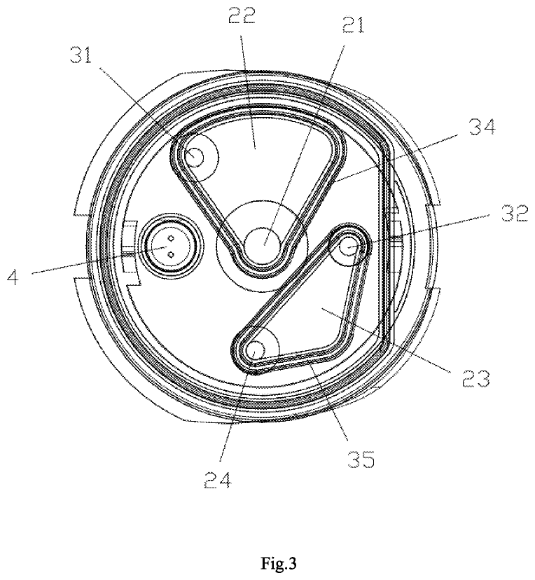

Furthermore, as shown in FIG. 3, the first airflow chamber 22 extends in its length direction, and the air-suctioning hole 21 and the air-suctioning end 31 are respectively arranged at the two ends of the first airflow chamber 22 in its length direction.

In this embodiment, the air-expelling hole 24 is arranged on one side of the top wall of the second airflow chamber 23, and the air-expelling end 32 is arranged on one side of the bottom wall of the second airflow chamber 23; thus, by way of arranging the air-expelling hole 24 and the air-expelling end 32 at different positions of the second airflow chamber 23, it is possible for the foreign matter in spite of continuously flowing to stay in the second airflow chamber 23 on flowing thereinto, ensuring the smoothness of the air passage.

In this embodiment, an installation groove 11 is disposed inside the fixing casing 1, and the air passage piece 2 is accommodated in the installation groove 11; when installing the air passage piece 2, the air passage piece 2 is directly embedded into the installation groove 11, and then the conversion cap 3 is fitted thereon; thus, finishing the installation.

Furthermore, as shown in FIG. 3, the second airflow chamber 23 extends in its length direction, and the air-expelling hole 24 and the air-expelling end 32 are respectively arranged at both ends of the second airflow chamber 23 in its length direction.

In this embodiment, a transition groove 12 is disposed at one end of the fixing casing 1, and the air-suctioning hole 21 and the air-expelling hole 24 both communicate with the transition groove 12; when the suctioning tip assembly 6 is mounted on the fixing casing 1, one end of the suctioning tip assembly 6 directly fixedly fits into the transition groove 12.

In this embodiment, a plug-in lip 25 is disposed at one end of the air passage piece 2, and the plug-in lip 25 penetrates the fixing casing 1. The air-suctioning hole 21 communicates with the transition groove 12 through the plug-in lip 25. When installing the suctioning tip assembly 6, a connecting tube on the suctioning tip assembly 6 directly fits with the plug-in lip 25, so that the suctioning tip assembly 6 communicates with the air-suctioning hole 21. After the air pump 5 starts, a negative pressure is transmitted from the air-suctioning hole 21 to the suctioning tip assembly 6.

In this embodiment, a first evading hole 13 is disposed on the fixing casing 1, and the air-expelling hole 24 communicates with the transition groove 12 through the first evading hole 13. After the air pump 5 starts, the air-expelling hole 24 expels air into the transition groove 12 through the first evading hole 13.

In this embodiment, an embedded ring groove 14 is disposed at one end of the fixing casing, and an embedding protrusion ring 33 is disposed on one side of the conversion cap 3. The embedding protrusion ring 33 fits into the embedded ring groove 14 and makes a tight join on the wall of the embedded ring groove 14. When the conversion cap 3 fits onto the fixing casing 1, in order to enable the air passage piece 2 to be airtightly installed inside the fixing casing 1, the way of making the embedding protrusion ring 33 tightly fit into the embedded ring groove 14 improves the airtightness level after installation.

In this embodiment, since the air passage piece 2 is made from silicone materials, in order to enhance the airtightness of the first airflow chamber 22 and the second airflow chamber 23, and to avoid decrease in negative pressure caused by air leakage between the first airflow chamber 22 and the second airflow chamber 23, and cross-flow occurring between the first airflow chamber 22 and the second airflow chamber 23, a first sealing ring portion 34 and a second sealing ring portion 35 are arranged on the conversion cap 3. The first sealing ring portion 34 and the second sealing ring portion 35 are similar in structure, both have an annular protruding structure. The first sealing ring portion 34 or the second sealing ring portion 35 may be formed by stacking multiple layers or single layer of annular protruding structures outward expansion in sequence. The first sealing ring portion 34 lies against the surface of the air passage piece 2 and surrounds the periphery of the first airflow chamber 22, so that the first airflow chamber 22 is covered and sealed by the conversion cap 3. The second sealing ring portion 35 lies against the surface of the air passage piece 2 and surrounds the periphery of the second airflow chamber 23, so that the second airflow chamber 23 is covered and sealed by the conversion cap 3. This design ensures that there is no air leakage or cross-flow between the first airflow chamber 22 and the second airflow chamber 23.

In this embodiment, the filter air passage assembly for nasal aspirators further comprises an illuminating bead 4 and an accommodating groove 26 disposed on the air passage piece 2. The illuminating bead 4 is embedded in the accommodating groove 26. A second evading hole 15 is disposed on the fixing housing 1, and the second evading hole 15 communicates with the transition groove 12, and the illuminating bead 4 is aligned with the second evading hole 15. The illuminating bead 4 is electrically connected with the main body of the nasal aspirator. During using the nasal aspirator, users turn on the illuminating bead, so as to illuminate the interior of the suctioning tip assembly 6 to observe the volume of mucus inside the suctioning tip assembly 6.

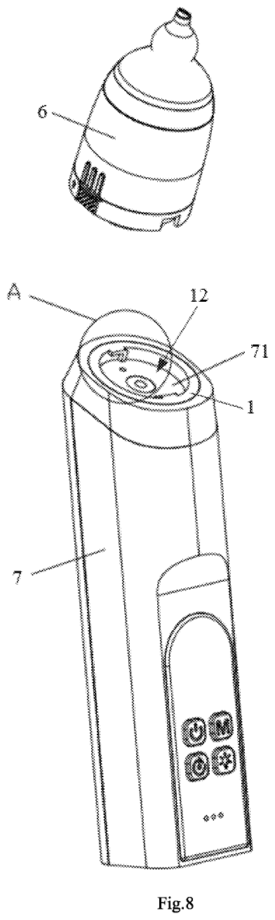

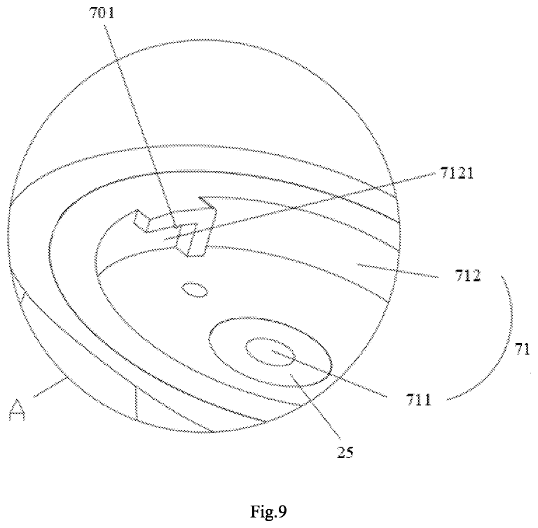

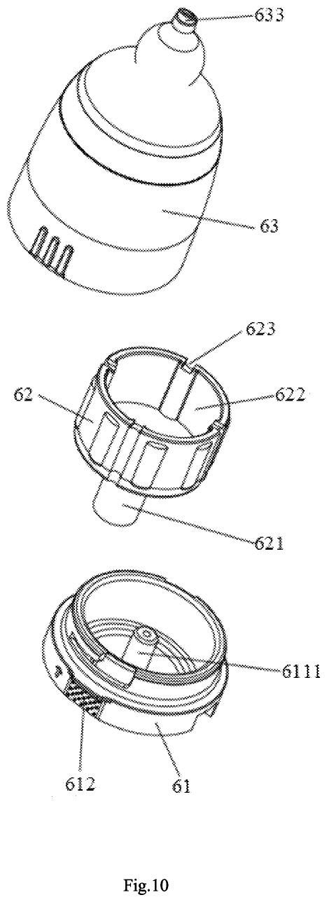

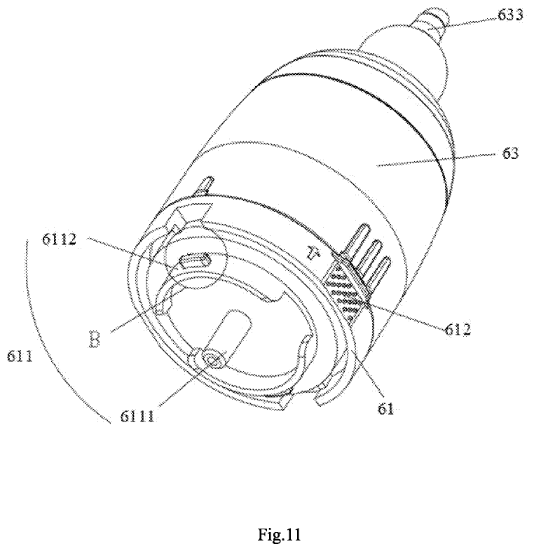

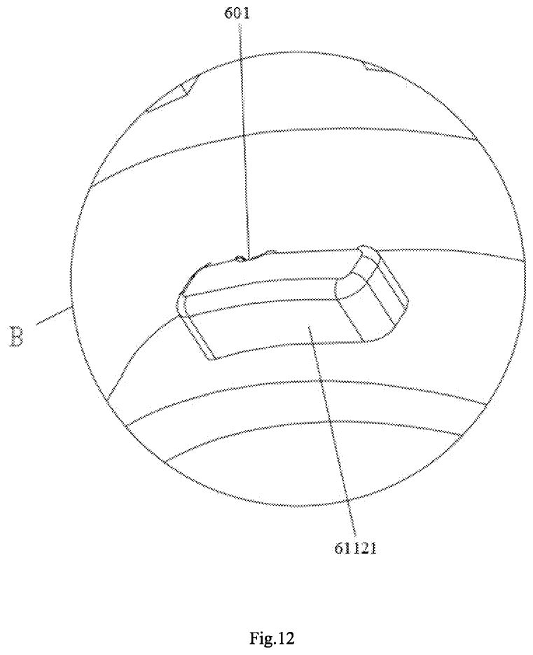

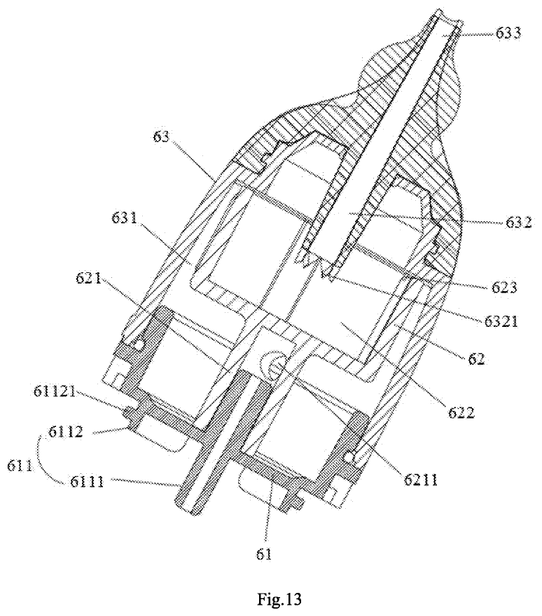

As shown in FIGS. 8-13, the present invention proposes an easily-replaceable integrated suctioning tip assembly, comprising a main body 7 and a suctioning tip assembly 6. One end of the suctioning tip assembly 6 is detachably connected and communicates with one end of the main body 7. The suctioning tip assembly 6 includes a connecting communicating seat 61, a collecting piece 62, and a suction nozzle 63. The main body 7 is the body of the nasal aspirator, inside which a circuit board, a battery, and a vacuum pump are arranged. The circuit board sends instructions to a vacuum pump through its operating program, so as to control the vacuum pump to pump air. The battery provides electrical energy for the operation of both the circuit board and the vacuum pump. One end of the connecting communicating seat 61 fits and communicates with one end of the main body 7. The other end of the connecting communicating seat 61 is detachably connected and communicates with the collecting piece 62. One end of the suction nozzle 63 forms a detachable sealed connection with the periphery of the connecting communicating seat 61. The suction nozzle 63 envelops the collecting piece 62 to form a negative pressure space 631, and the suction nozzle 63 communicates with the negative pressure space 631.

In this embodiment, by way of providing the suctioning tip assembly 6 composed of the connecting communicating seat 61, the collecting piece 62, and the suction nozzle 63 and the detachable connection formed by the connecting communicating seat 61 and the main body 7, it is possible for users to directly suction foreign matter such as mucus into the suctioning tip assembly 6 for storage, specifically into the collecting piece 62. Even when the foreign matter such as mucus overflows, it will fall into the connecting communicating seat 61 and will not flow into the main body 7. When it is necessary to clean the suctioning tip assembly 6, users can directly detach the suctioning tip assembly 6 from the main body 7. Since the suctioning tip assembly 6 is an integrated unit, the collected mucus falls into the suctioning tip assembly 6, preventing contamination on the main body 7. When cleaning the interior of the suctioning tip assembly 6, users can directly disassemble the suctioning tip assembly 6 into the connecting communicating seat 61, the collecting piece 62, and the suction nozzle 63 for respective cleaning, with advantages such as convenience and usefulness.

In summary, the easily-replaceable integrated suctioning tip assembly can be disassembled together with a part connected with a suctioning tip, making subsequent cleaning more convenient.

In this embodiment, a mounting portion 611 is disposed at one end of the connecting communicating seat 61, and an engagement ventilation portion 71 is disposed at one end of the main body 7. The mounting portion 611 is accommodated in the engagement ventilation portion 71 and forms an engagement connection with the engagement ventilation portion 71, and the mounting portion 611 communicates with the engagement ventilation portion 71. When installing the suctioning tip assembly 6, users can align the mounting portion 611 with the engagement ventilation portion 71 and press it into the engagement ventilation portion 71, then turn the mounting portion 611 by a preset angle, so as to form an engagement connection with the engagement ventilation portion 71 for fixation, so that the suctioning tip assembly 6 is mounted on the main body 7 to form a conduit.

In this embodiment, the mounting portion 611 is provided with an inserted conduit column 6111, and the engagement ventilation portion 71 is provided with an air-suctioning groove 711. One end of the inserted conduit column 6111 is accommodated in the air-suctioning groove 711, and the outer wall of the inserted conduit column 6111 closely fits onto the wall of the air-suctioning groove 711. After the suctioning tip assembly 6 is successfully joined to the main body 7, the inserted conduit column 6111 is inserted into the air-suctioning groove 711 and forms an airtight connection. When suction occurs to the main body 7, a negative pressure is transmitted to the negative pressure space 631 through the inserted conduit column 6111.

In this embodiment, a plug-in sleeve 621 is disposed on the collecting piece 62. The plug-in sleeve 621 forms a detachable connection and communicates with the other end of the inserted conduit column 6111. A through hole 6211 is disposed on one side of the plug-in sleeve 621, and the through hole 6211 communicates with the negative pressure space 631. When assembling the suctioning tip assembly 6, users can mount the collecting piece 62 on the inserted conduit column 6111 through the plug-in sleeve 621, then fit the suction nozzle 63 with the connecting communicating seat 61. After the main body 7 is opened, a negative pressure is transmitted through the inserted conduit column 6111 into the plug-in sleeve 621, and then through the through hole 6211 into the negative pressure space 631.

In this embodiment, the mounting portion 611 is provided with a caught side ring 6112, and the engagement ventilation portion 71 is provided with an embedded catch groove 712. The caught side ring 6112 fits into the embedded catch groove 712 and forms an engagement connection with the wall of the embedded catch groove 712. When fitting the suctioning tip assembly 6 with the main body 7, users can insert the caught side ring 6112 into the embedded catch groove 712, then turn the suctioning tip assembly 6, so as to enable the caught side ring 6112 to fit onto the inner wall of the embedded catch groove 712 and rotate, thereby forming an engagement connection between the caught side ring 6112 and the embedded catch groove 712.

In this embodiment, a caught plate 61121 is disposed on the periphery of the caught side ring 6112, and a catch opening 7121 is disposed on the wall of the embedded catch groove 712. The caught plate 61121 fits into the catch opening 7121. When fitting the caught side ring 6112 with the embedded catch groove 712, users turn the suctioning tip assembly 6, so as to make the caught plate 61121 slide along the wall of the embedded catch groove 712 into the catch opening 7121 to form a engagement connection.

In this embodiment, a positioning groove 601 is disposed on the caught plate 61121, and the catch opening 7121 is provided with a positioning protrusion structure 701, which is accommodated within the positioning groove 601. In order to ensure that the caught plate 61121 forms a more stable engagement connection with the catch opening 7121, the positioning protrusion structure fits with the positioning groove 601 to increase the fixing force.

In this embodiment, a suction tube 632 is disposed inside the suction nozzle 63, and a collecting groove 622 is disposed at one end of the collecting piece 62. One end of the suction tube 632 extends into the collecting groove 622. A plurality of guiding grooves 623 are disposed at one end of the collecting piece 62, and circumferentially uniformly distributed. The plurality of guiding grooves 623 communicate with the collecting groove 622, and also communicate with the negative pressure space 631. The suction tube 632 communicates with the negative pressure space 631 through the plurality of guiding grooves 623. After the main body 7 starts to generate a negative pressure, the negative pressure gets into the negative pressure space 631, then sequentially passes through the guiding groove 623 and the collecting groove 622 into the suction tube 632, and then by means of transmission through the suction tube 632, a suction nozzle end 633 of the suction nozzle 63 generates a negative pressure. When the suction nozzle end 633 is inserted into a user's nostril for suction, mucus is drawn out by the suction nozzle end 633, then gets into the suction tube 632, and finally the mucus is drained from the suction tube 632 into the collecting groove 622 for storage.

In this embodiment, a plurality of collecting tips 6321 are disposed at one end of the suction tube 632, and extend into the collecting groove 622; when draining mucus, the plurality of collecting tips 6321 make it easier for the mucus to fall into the collecting groove 622, raising its effectiveness.

In this embodiment, an anti-slip portion 612 for improving grasp strength is disposed on the periphery of the connecting communicating seat 61. The anti-slip portion 612 makes it easy for users to grasp the connecting communicating seat 61 and turn it forward to form an engagement connection with the main body 7, and also makes it easy for users to grasp the connecting communicating seat 61 to turn it backward, so that the suctioning tip assembly 6 rotates in reverse and disengages from the main body 7.

The present invention also proposes a nasal aspirator, comprising a main body 7, a filter air passage assembly, and a suctioning tip assembly 6. The specific structure of the filter air passage assembly refers to all the embodiments of the filter air passage assembly for nasal aspirators described above. The specific structure of the suctioning tip assembly 6 refers to all embodiments of the easily-replaceable integrated suctioning tip assembly described above. Since this nasal aspirator adopts all the technical solutions of the aforementioned embodiments, it at least has all the beneficial effects brought by the technical solutions of the aforementioned embodiments, which will not be repeated herein.

Of course, the present invention may also have various other embodiments. Based on this embodiment, any other embodiments obtained by a person skilled in the art without inventive work fall within the protection scope of the present invention.

What is claimed is:

1. A filter air passage assembly for nasal aspirators, configured to be fixedly mounted on one end of a main body of the nasal aspirator, and joined to a suctioning tip assembly;
comprising a fixing casing, an air passage piece, and a conversion cap;
wherein the air passage piece is fixedly connected inside the fixing casing, and the conversion cap forms a sealed and fixed connection together with the fixing casing and covers the air passage piece;
wherein an air-suctioning hole, a first airflow chamber, a second airflow chamber, and an air-expelling hole are disposed on the air passage piece, and the air-suctioning hole and the air-expelling hole both communicate with external environments through the fixing casing, and the air-suctioning hole communicates with the first airflow chamber, and the air-expelling hole communicates with the second airflow chamber;
wherein an air-suctioning end and an air-expelling end are disposed on the conversion cap, and the air-suctioning end communicates with the first airflow chamber and staggers the air-suctioning hole, and the air-expelling end communicates with the second airflow chamber and staggers the air-expelling hole.

2. The filter air passage assembly for nasal aspirators according to claim 1, wherein the air-suctioning hole is arranged on one side of the top wall of the first airflow chamber, while the air-suctioning end is arranged on one side of the bottom wall of the first airflow chamber; or, the air-expelling hole is arranged on one side of the top wall of the second airflow chamber, while the air-expelling end is arranged on one side of the bottom wall of the second airflow chamber; or
wherein the air-suctioning hole is arranged on one side of the top wall of the first airflow chamber, while the air-suctioning end is arranged on one side of the bottom wall of the first airflow chamber; and, the air-expelling hole is arranged on one side of the top wall of the second airflow chamber, while the air-expelling end is arranged on one side of the bottom wall of the second airflow chamber.

3. The filter air passage assembly for nasal aspirators according to claim 1, wherein the volume of the first airflow chamber is a, while the volume of the second airflow chamber is b, conforming to a>b.

4. The filter air passage assembly for nasal aspirators according to claim 1, wherein an installation groove is disposed at one end of the fixing casing, and the air passage piece is accommodated in the installation groove; a transition groove is disposed at the other end of the fixing casing, and configured to mount one end of the suctioning tip assembly, and the air-suctioning hole and the air-expelling hole both communicate with the transition groove.

5. The filter air passage assembly for nasal aspirators according to claim 4, wherein a plug-in lip is disposed at one end of the air passage piece, and the plug-in lip penetrates the fixing casing, and the air-suctioning hole communicates with the transition groove through the plug-in lip.

6. The filter air passage assembly for nasal aspirators according to claim 1, wherein an embedded ring groove is disposed at one end of the fixing casing, and an embedding protrusion ring is disposed on one side of the conversion cap, and the embedding protrusion ring fits into the embedded ring groove and makes a tight join on the wall of the embedded ring groove.

7. The filter air passage assembly for nasal aspirators according to claim 1, wherein the filter air passage assembly for nasal aspirators further comprises an illuminating bead and an accommodating groove disposed on the air passage piece, and the illuminating bead is embedded in the accommodating groove, and a second evading hole is disposed on the fixing housing, and the second evading hole communicates with a transition groove, and the illuminating bead is aligned with the second evading hole.

8. The filter air passage assembly for nasal aspirators according to claim 1, wherein the air passage piece is made from soft materials.

9. The filter air passage assembly for nasal aspirators according to claim 2, wherein the first airflow chamber extends in its length direction, and the air-suctioning hole and the air-suctioning end are respectively arranged at the two ends of the first airflow chamber in its length direction; or the second airflow chamber extends in its length direction, and the air-expelling hole and the air-expelling end are respectively arranged at the two ends of the second airflow chamber in its length direction; or
wherein the first airflow chamber extends in its length direction, and the air-suctioning hole and the air-suctioning end are respectively arranged at the two ends of the first airflow chamber in its length direction; and the second airflow chamber extends in its length direction, and the air-expelling hole and the air-expelling end are respectively arranged at the two ends of the second airflow chamber in its length direction.

10. The filter air passage assembly for nasal aspirators according to claim 1, wherein the filter air passage assembly for nasal aspirators is configured to be connected with an easily-replaceable integrated suctioning tip assembly, which comprises a main body and a suctioning tip assembly;
one end of the suctioning tip assembly is detachably connected and communicates with one end of the main body, and the suctioning tip assembly includes a connecting communicating seat, a collecting piece, and a suction nozzle; one end of the connecting communicating seat fits and communicates with one end of the main body, and the other end of the connecting communicating seat is detachably connected and communicates with the collecting piece; one end of the suction nozzle forms a detachable sealed connection with the periphery of the connecting communicating seat, and the suction nozzle envelops the collecting piece to form a negative pressure space, and the suction nozzle communicates with the negative pressure space, so as to make it possible to store foreign matter suctioned by the suctioning tip assembly in the collecting piece, and ensure that when the foreign matter in the collecting piece overflows, it falls into the connecting communicating seat.

11. A nasal aspirator, comprising a main body, a suctioning tip assembly and a filter air passage assembly having a fixing casing, an air passage piece, and a conversion cap;
wherein the air passage piece is fixedly connected inside the fixing casing, and the conversion cap forms a sealed and fixed connection together with the fixing casing and covers the air passage piece;
wherein an air-suctioning hole, a first airflow chamber, a second airflow chamber, and an air-expelling hole are disposed on the air passage piece, and the air-suctioning hole and the air-expelling hole both communicate with external environments through the fixing casing, and the air-suctioning hole communicates with the first airflow chamber, and the air-expelling hole communicates with the second airflow chamber;
wherein an air-suctioning end and an air-expelling end are disposed on the conversion cap, and the air-suctioning end communicates with the first airflow chamber and staggers the air-suctioning hole, and the air-expelling end communicates with the second airflow chamber and staggers the air-expelling hole;
wherein the fixing casing is fixedly mounted on one end of the main body, and the fixing casing is configured as a structure connected with the suctioning tip assembly;
wherein the suctioning tip assembly communicates with the air passage piece, and an air pump is disposed inside the main body, and the air-suctioning end and the air-expelling end respectively communicate with an air-suctioning port and an air-expelling port of the air pump.

12. The nasal aspirator according to claim 11, wherein one end of the suctioning tip assembly is detachably connected and communicates with one end of the main body, and the suctioning tip assembly includes a connecting communicating seat, a collecting piece, and a suction nozzle; one end of the connecting communicating seat fits and communicates with one end of the main body, and the other end of the connecting communicating seat is detachably connected and communicates with the collecting piece; one end of the suction nozzle forms a detachable sealed connection with the periphery of the connecting communicating seat, and the suction nozzle envelops the collecting piece to form a negative pressure space, and the suction nozzle communicates with the negative pressure space, so as to make it possible to store foreign matter suctioned by the suctioning tip assembly in the collecting piece, and ensure that when the foreign matter in the collecting piece overflows, it falls into the connecting communicating seat.

13. The nasal aspirator according to claim 12, wherein a mounting portion is disposed at one end of the connecting communicating seat, and an engagement ventilation portion is disposed at one end of the main body, and the mounting portion is accommodated in the engagement ventilation portion and forms an engagement connection with the engagement ventilation portion, and the mounting portion communicates with the engagement ventilation portion.

14. The nasal aspirator according to claim 13, wherein the mounting portion is provided with an inserted conduit column, and the engagement ventilation portion is provided with an air-suctioning groove, and one end of the inserted conduit column is accommodated in the air-suctioning groove, and the outer wall of the inserted conduit column closely fits onto the wall of the air-suctioning groove.

15. The nasal aspirator according to claim 14, wherein a plug-in sleeve is disposed on the collecting piece, and the plug-in sleeve forms a detachable connection and communicates with the other end of the inserted conduit column, and a through hole is disposed on a side wall of the plug-in sleeve, and the through hole communicates with the negative pressure space.

16. The nasal aspirator according to claim 13, wherein the mounting portion is provided with a caught side ring, and the engagement ventilation portion is provided with an embedded catch groove, and the caught side ring fits into the embedded catch groove and forms an engagement connection with the wall of the embedded catch groove.

17. The nasal aspirator according to claim 16, wherein a caught plate is disposed on the periphery of the caught side ring, and a catch opening is disposed on the wall of the embedded catch groove, and the caught plate fits into the catch opening.

18. The nasal aspirator according to claim 12, wherein a suction tube is disposed inside the suction nozzle, and a collecting groove is disposed at one end of the collecting piece, and one end of the suction tube extends into the collecting groove, and a plurality of guiding grooves are disposed at one end of the collecting piece, and circumferentially uniformly distributed, and the plurality of guiding grooves communicate with the collecting groove, and also communicate with the negative pressure space, and the suction tube communicates with the negative pressure space through the plurality of guiding grooves.

19. The nasal aspirator according to claim 18, wherein a plurality of collecting tips are disposed at one end of the suction tube, and extend into the collecting groove.

20. The nasal aspirator according to claim 12, wherein an anti-slip portion for improving grip strength is disposed on the periphery of the connecting communicating seat.
